SEQUENCE LISTING (28 sequences)

SEQ ID NO: 1 - RNA, 21 nt, EHV-1
ggauggagacuuuuacaccuu

SEQ ID NO: 2 - RNA, 21 nt, EHV-1
gguguaaaagucuccauccuc

SEQ ID NO: 3 - RNA, 21 nt, EHV-1
ggagaacgagauuuucacguu

SEQ ID NO: 4 - RNA, 21 nt, EHV-1
cgugaaaaucucguucuccuc

SEQ ID NO: 5 - RNA, 21 nt, EHV-1
cggaaaucgagguuaucaguu

SEQ ID NO: 6 - RNA, 21 nt, EHV-1
cugauaaccucgauuuccgug

SEQ ID NO: 7 - RNA, 21 nt, EHV-1
cgauaaccuccucaacaauuu

SEQ ID NO: 8 - RNA, 21 nt, EHV-1
auuguugaggagguuaucguc

SEQ ID NO: 9 - RNA, 21 nt, EHV-1
cgaugguucaccucaacaauu

SEQ ID NO: 10 - RNA, 21 nt, EHV-1
uuguugaggugaaccaucgua

SEQ ID NO: 11 - RNA, 21 nt, EHV-1
cggagguuuuugaaaacgauu

SEQ ID NO: 12 - RNA, 21 nt, EHV-1
ucguuuucaaaaaccuccguc

SEQ ID NO: 13 - DNA, 22 bp, EHV-1
gcgaagtacccctcgttcatct

SEQ ID NO: 14 - DNA, 19 bp, EHV-1
atgctcgggcgctcctact

SEQ ID NO: 15 - DNA, 23 bp, EHV-1
cgctgaggatggagacttttaca

SEQ ID NO: 16 - DNA, 18 bp, EHV-1
ggtggttcgatgcgtacg

SEQ ID NO: 17 - DNA, 20 bp, EHV-1
tggtaacggtgggccttagt

SEQ ID NO: 18 - DNA, 20 bp, EHV-1
gggcttgacgtaggcaaaca

SEQ ID NO: 19 - DNA, 18 bp, Rabbit
cgagatcgtgcgggacat

SEQ ID NO: 20 - DNA, 20 bp, Rabbit
gccatctcctgctcgaagtc

SEQ ID NO: 21 - DNA, 14 bp, Artificial sequence
  control probe
tcgcgacaccgcct

SEQ ID NO: 22 - DNA, 21 bp, Artificial sequence
  control probe
ccaccgcctaccggatccacc

SEQ ID NO: 23 - DNA, 23 bp, Artificial sequence
  control probe
ttgatacggctcatttccacagc

SEQ ID NO: 24 - DNA, 26 bp, Artificial sequence
  control probe
aaggagaagctgtgctacgtggcgct

SEQ ID NO: 25 - DNA, 22 bp, EHV-1
tttggaagctgtgttgttagag

SEQ ID NO: 26 - DNA, 21 bp, EHV-1
taatcactgcggtattgtcca

SEQ ID NO: 27 - DNA, 20 bp, EHV-1
cacttgcaccagccacgttc

SEQ ID NO: 28 - DNA, 20 bp, EHV-1
catgggggtaaagatgggct

(19) United States
(12) Patent Application Publication
(10) Pub. No.: US 2011/0182865 A1
(43) Pub. Date: Jul. 28, 2011

(54) Method for Prophylaxis and Treatment of Equine Herpesvirus Type 1 Infections

(76) Inventors: Nikolaus Osterrieder, Potsdam (DE); Gerlinde Van De Walle, Lemberge (BE); Sarah T. Peters, Avondale, PA (US)

(21) Appl. No.: 12/866,539

(22) PCT Filed: Feb. 9, 2009
(86) PCT No.: PCT/US09/33537

§ 371 Date: Apr. 8, 2011

Related U.S. Application Data
(60) Provisional application No. 61/027,233, filed on Feb. 8, 2008.

(51) Int. Cl. A61K 31/7052; A61K 35/76; A61P 31/12

(52) U.S. Cl. 424/93.6; 514/44 A

(57) ABSTRACT

Provided are compositions and methods for treatment and/or prophylaxis of EHV-I infections in horses. The compositions and methods effect treatment and/or prophylaxis of EHV-I infections through RNAi mediated inhibition of EHV-I gB and EHV-I Ori genes, which results in a reduction of the severity of neurological symptoms that are induced by EHV-I infection in horses, and/or a reduction in EHV-I viral shedding in the horses. Included are siRNAs or shRNAs that are designed to target EHV-I gB and EHV-I Ori helicase mRNAs. Also included are vectors encoding such shRNAs.

This application claims priority to U.S. application Ser. No. 61/027,233, filed on Feb. 8, 2008, the disclosure of which is incorporated herein by reference.

### FIELD OF THE INVENTION

The present invention relates generally to the field of animal health, and in particular to compositions and methods for the prophylaxis and treatment of equine herpesvirus type 1 infections in horses.

### BACKGROUND OF THE INVENTION

Equine herpesvirus type 1 (EHV-1) is a major cause of respiratory, neurologic and reproductive disease in horses worldwide. EHV-1 is a member of the Alphaherpesvirinae and closely related to the causative agents of human chicken pox/shingles (varicella zoster virus, VZV) as well as cold sores and genital herpes, herpes simplex types 1 (HSV-1) and 2 (HSV-2). EHV-1 is spread through respiratory secretions and replicates in the nasal epithelium upon gaining access to the respiratory tract. Initial replication is followed by a leukocyte-associated viremia. Further replication of EHV-1 can occur in endothelia of blood vessels of the central nervous system (CNS) and the uterus, where vasculitis can lead to myeloencephalopathy or abortion, respectively. EHV-1 establishes latency in neuronal and lymphoid tissue and recrudesces in times of stress such as transportation, pregnancy, competitions, and racing. Therefore, horses traveling to competitions and coming into contact with new animals are at risk of shedding or contracting the virus, making this viral infection a significant concern for the performance horse industry. Recently, an increased occurrence of EHV-1 outbreaks, especially of the neurologic form of the disease, has been reported, and these outbreaks are more and more frequently associated with high mortality rates. Consequently, EHV-1 has recently been classified as a potentially emerging infectious disease by the US Department of Agriculture.

Vaccination is currently the only form of EHV-1 control but the efficacy of the available vaccines is questionable. As EHV-1-induced protective immunity is only short-lived, vaccination has to be repeated at least every 6 months [Kydd J H, et al. Vet Immunol Immunopathol 2006 May 15; 111(1-2):15-30]. However, the continuing EHV-1 outbreaks involving large numbers of animals raises the question as to whether repeated vaccination by itself is sufficient to protect animals in outbreak situations. Moreover, repeated vaccination has recently been suggested to predispose horses to develop the most severe form of EHV-1 infection, myeloencephalopathy [Kydd J H, et al. Vet Immunol Immunopathol 2006 May 15; 111(1-2):15-30; Goodman L B, et al. Vaccine 2006 Apr. 24; 24(17):3636-45; Henninger R W, et al. J Vet Intern Med 2007 January; 21(1):157-65]. Further, experimental antiviral drugs have yet to demonstrate proven clinical efficacy. For example, drugs such as acyclovir or valacyclovir were shown to have serious limitations due to poor bioavailability, and high therapeutic concentrations are needed in order to reach the desired effect, making these drugs a costly medication with questionable efficacy. Thus, there is an ongoing need for compositions and methods for improved treatment and/or prophylaxis of EHV-1 infections in horses.

### SUMMARY OF THE INVENTION

The present invention provides compositions and methods for treatment and/or prophylaxis of EHV-1 infections in horses. In particular, the invention provides for reducing the severity of neurological symptoms that are induced by EHV-1 infection in horses. Such symptoms include ataxia (e.g., stumbling while walking), difficulty or inability to stand (recumbence), mild, moderate or complete paralysis, and in some instances, an inability to pass urine. The invention also provides for a reduction in EHV-1 viral shedding in horses after exposure to EHV-1.

The method of the invention involves suppressing expression of EHV-1 glycoprotein B (gB), and/or EHV-1 origin-binding protein (Ori) helicase via RNAi (RNA interference) based methods. EHV-1 gB is an envelope protein that is essential for EHV-1 entry into cells and for cell-to-cell spread [Neubauer et al. Virology 1997 Jan. 20; 227(2):281-94]. EHV-1 Ori helicase is an enzyme necessary for EHV-1 genome replication [Martin D W, et al. J Virol 1994 June; 68(6):3674-81]. The viral genomic gB and On helicase DNA and mRNA coding sequences are known in the art. In particular, the EHV-1 genomic sequence is accessible at Genbank Sequence #AY665713 (Sep. 8, 2005 entry), which also provides predicted amino acid sequences based on open reading frames (ORFs). EHV-1 ORF33 encodes gB and ORF53 encodes the Ori helicase.

The method comprises administering to a horse a composition comprising polynucleotides that are capable of suppressing expression of EHV-1 gB, and/or polynucleotides that are capable of suppressing expression of EHV-1 Ori helicase. Representative polynucleotide sequences that can be used in performance of the method are provided in Table 1.

It is preferable to administer simultaneously a composition comprising siRNA or shRNA that is capable of inhibiting expression of EHV-1 gB, in combination with another siRNA or shRNA that is capable of inhibiting expression of EHV-1 Ori helicase. The method also includes administering vectors that encode polynucleotides that are capable of inhibiting expression of EHV-1 gB, and/or polynucleotides that are capable of inhibiting expression of EHV-1 Ori helicase. In one embodiment, the vectors used in the method encode shRNA.

The invention also provides compositions comprising the aforementioned polynucleotides. The polynucleotides present in the compositions can be in the form of siRNAs, shRNAs, or vectors that encode shRNAs.

Polynucleotides that can be used in the invention can comprise RNA, RNA:DNA hybrids, or other modifications that can improve the capability of the polynucleotides to resist endonuclease degradation and/or cleave their target EHV-1 gene products. The polynucleotides can be administered to horses without pharmaceutical carriers, or as a pharmaceutical preparation, or by a administering a recombinant vector that provides for transcription of polynucleotide sequences targeted to EHV-1 gB, and/or EHV-1 Ori helicase mRNA.

In one embodiment, the polynucleotides are administered via intranasal administration, but the administration may be performed via any other suitable route. The polynucleotides can be delivered as a single or in multiple doses. The polynucleotides can be administered prior to exposure to EHV-1, during exposure, after exposure, or in any combination of such time points as required by the duration and severity of a specific disease outbreak. In general, the administrations can be performed up to three days before an anticipated exposure, during an exposure, and/or for three days after symptoms of infection are no longer apparent.

The compositions and method of the invention are shown to be capable of inhibiting EHV-1 infection in vitro as well as in a murine model of EHV-1 infection. It is also demonstrated that the invention can reduce the incidence and severity of neurological symptoms that are induced by EHV-1 infection in horses, as well as a reduction in viral shedding. Thus, the invention provides useful compositions and methods for prophylaxis, metaphylaxis and treatment of EHV-1 infections.

### BRIEF DESCRIPTION OF THE FIGURES

FIGS. 1A through 1C provide graphical representations and images showing the effect of siRNAs targeting glycoprotein B (gB) and origin-binding protein (ori) helicase on EHV-1 replication and cell-to-cell spread. For FIG. 1A, siRNA's targeting gB (sigB3) or Ori (SiOri2) were transfected into RK13 cells and cells were infected with 100 PFU of EHV-1 strain rAb4Δgp2 14 h later. Supernatants were collected 24 h p.i. from sigB3-(□) and SiOri2-transfected cells (⋄), and viral titers were determined with standard plaque assays. Asterisks indicate statistically significant differences (*: p<0.05, **: p<0.01). For FIG. 1B, cells were fixed with 10% formalin and the average plaque areas were determined. Asterisks indicate statistically significant differences (p<0.05). For FIG. 1C, control cells were included, which were either not transfected with siRNA or were transfected with 75 pmol siGFP or siLuc before infection with rAb4Δgp2. Representative pictures were taken with light and fluorescent microscopy (FIG. 1C, panel a) and viral titers were determined with standard plaque assays (FIG. 1C, panel b).

FIGS. 2A and 2B provide graphical and photographic data showing that siRNAs efficiently suppress gB and Ori expression at the mRNA and protein level. For FIG. 2A, relative qRT-PCR was performed. RK13 cells were transfected with 37.5 pmol sigB3, 75 pmol SiOri2, 75 pmol siLuc or not transfected. Cells were infected 14 h later with 500 PFU of rAb4Δgp2 and at 12 h p.i., RNA was extracted from infected cells. RT-PCR was used to determine relative levels of gB or Ori (white bars) or EHV-1 IR6 (grey bars) mRNA, using rabbit β-actin as the endogenous housekeeping gene. Asterisks indicate statistically significant differences (p<0.05). FIG. 2B shows Western blot analysis. RK13 cells were either not transfected or transfected with 75 pmol of the control siRNA siLuc or 75 pmol sigB3. Cells were infected 14 h later with 500 PFU of rAb4Δgp2. At 24 h p.i., cell lysates were prepared and analyzed by SDS-PAGE under reducing conditions. Anti-gB mAb 3F6 (1/500) and anti-β-actin (1/5000), followed by anti-mouse IgG peroxidase (1/7500) were used. Cell lysates from non-infected RK13 cells were included as a control.

FIGS. 3A and 3B provide graphical representations of data showing that combining siRNA's have a greater than additive effect on reduction of EHV-1 replication and are effective before and after infection. For FIG. 3A, the siRNA's sigB3 and SiOri2 were transfected into RK13 cells, either alone or in different combinations, and cells were infected 14 h later with 500 PFU of rAb4Δgp2. Supernatants were collected 24 h p.i. and viral titers were determined by standard plaque assays. Asterisks indicate statistically significant differences (p<0.05). For FIG. 3B, RK13 cells were transfected with 75 pmol control siRNA (black bars), 37.5 pmol sigB3 (grey bars), 75 pmol SiOri2 (hatched bars) or a combination of 6.25 pmol sigB3 and 6.25 pmol SiOri2 (white bars). At different times after transfection (ranging form 12 h up to 0 h) cells were infected with 500 PFU of rAb4Δgp2 and supernatants were collected at 24 h p.i. In one set of experiments, cells were first infected with rAb4Δgp2 and transfected with the different siRNA's 1 h later. Viral titers were determined with standard plaque assays. Asterisks indicate statistically significant differences (p<0.05).

FIGS. 4A-4C provide graphical and photographic data showing that siRNAs are effective in reducing inflammatory responses in a murine model of EHV-1 infection when applied 0.5 h before infection. FIGS. 4A and 4B summarize development of mean body weights after infection. Balb/c mice (groups of 12) were transfected intranasally with sigB3 and SiOri2, alone or in combination, and mice were infected intranasally with 1×10⁵ PFU of Ab4 0.5 h later. Mice inoculated with 75 pmol siLuc were used as positive controls and uninfected negative control mice were also included. Mean body weights were determined on the day of infection (day 0) up to day 14 p.i. Mean body weights on the day of infection were set to 100%. Standard deviations (SD's) ranged from 0.7 to 2.8%. The day of the maximal SD's for each group are indicated in brackets. The days where statistically significant differences (SS) were observed, as determined with non-parametric Wilcoxon-Whitney and Kruskal-Wallis analyses, are also given between brackets. A total lung score based upon histopathological characteristics was determined at day 2 p.i. in three mice of each group. The score ranged from 0+ (normal) to 5+ (severe) as described in the Examples. Asterisks indicate statistically significant differences (p<0.05). FIG. 4C shows representative hematoxylin and eosin (H&E) images showing histological features in lungs of mice treated with the control siLuc and mice treated with siRNA's against EHV-1 genes. BH: bronchiolar hyperplasia, II: interstitial inflammation, BN: bronchiolar necrosis (FIG. 4C).

FIGS. 5A and 5B provide graphical data showing that siRNAs are effective in reducing viral replication when applied before infection, even in the absence of a transfection reagent. For FIG. 5A, Balb/c mice (groups of 12) were treated intranasally with sigB3 and SiOri2, alone or in combination, and mice were infected intranasally with 1×10⁵ PFU of Ab4 0.5 h later. For FIG. 5B, Balb/c mice (groups of 12) were inoculated intranasally with 62.5 pmol sigB3/SiOri2, complexed with lipofectamine (closed symbols) or in PBS (open symbols) 6 or 12 h before infection with 1×10⁵ PFU of Ab4. Mice transfected with 75 pmol siLuc were used as positive controls and viral titers were determined in three mice of each group on day 2 p.i. Titers in lungs and standard deviations are shown. Asterisks indicate statistically significant differences (p<0.05) between mice treated with sigB3 and SiOri2, alone or in combination, and mice inoculated with the control siRNA siLuc.

FIGS. 6A and 6B provide graphical representations of data showing that siRNAs are effective in reducing viral replication when applied after infection. Balb/c mice (groups of 15) were infected intranasally with 1×10⁵ PFU of Ab4. At 1, 6, 12 or 24 h post-infection, mice were inoculated intranasally with 62.5 pmol sigB3/SiOri2, in PBS. For FIG. 6A, viral titers were determined in lungs of five mice per group on day 3 p.i. by co-cultivation. Titers in lungs and standard deviations are shown. For FIG. 6B, viral loads in lung tissues were also measured by qPCR and are plotted as EHV-1 genome (IR6 gene) copies per million mouse iNOS gene copies. Asterisks indicate statistically significant differences (p<0.05; Student's t-test) between untreated mice and mice inoculated with 62.5 pmol sigB3/SiOri2.

FIG. 7 provides a graphical representation of data summarizing reductions in severity of neurological symptoms induced by EHV-1 infection in horses, relative to infected, untreated horses, by administering to the horses sigB3 and SiOri2 siRNA, the sequences of which are depicted in Table 1.

FIG. 8 provides a graphical representation of reduction in plaque forming units (PFU) (FIG. 8A) and virus genome copy numbers (FIG. 8B) in nasal swabs (NS) in horses treated with sigB3 and SiOri2 siRNA, relative to infected, untreated horses. The data presented in FIG. 8 excludes one horse treated with the sigB3/SiOri2 that exhibited a viremia of uncharacteristic early onset after challenge infection that may have been due to a non-experimentally induced EHV-1 recrudescence of latent virus or intercurrent disease caused by a different infectious agent.

### DESCRIPTION OF THE INVENTION

The present invention provides compositions and methods for treatment and/or prophylaxis of EHV-1 infections in horses. “Treatment” of a disease occurs when the severity of a symptom of the disease, the frequency with which such a symptom is exhibited by a horse, or both, is/are reduced or eliminated. For example, treatment of an EHV-1 virus infection can said to have occurred if a neurological symptom associated with the infection is alleviated or eliminated. “Prophylaxis” means completely or partially preventing EHV-1 infection or symptoms thereof. The invention may also be used for metaphylaxis, meaning performing the method on one or more horses at or near the time of a potential exposure to eliminate or minimize an expected outbreak of EHV-1 infection. Thus, metaphylaxis can be considered to be one type of prophylaxis.

The compositions and methods are used for RNAi mediated inhibition of EHV-1 gene expression. Performance of the method can result in a reduction of the severity of neurological symptoms that are induced by EHV-1 infection in horses, and/or a reduction in viral shedding and/or viral load in the circulation (viremia) in the horses.

Neurological symptoms induced by EHV-1 infection are well recognized in the art and include infected horses exhibiting ataxia (stumbling or difficulty in walking), recumbency, and mild, moderate or complete paralysis. An inability to pass urine can also be a neurological symptom induced by EHV-1 infection. Trained observers can score and categorize the severity of neurological symptoms induced by EHV-1 infection using standardized criteria known to those skilled in the art. (See, e.g., Equine Neurology, S. Reed and M. Furr, Blackwell Publishing, October 2007, p 66). The invention can also reduce the severity of the second peak of the biphasic fever characteristic of EHV-1 infection. In this regard, the normal body temperature of horses ranges from 99-101° F. The first fever spike can be identified by a body temperature over 103° F., and the second as a temperature of over 100° F.

The invention does not appear to adversely affect the humoral immune systems of horses, as determined by a greater than fourfold rise in mean titers in both treated and untreated horses, and the lack of a statistically significant difference in mean titers between the two groups.

The invention entails suppressing expression of EHV-1 gB and/or EHV-1 Ori by RNAi mediated gene suppression. The invention is accordingly considered to function by facilitating degradation of EHV-1 gB and EHV-1 Ori helicase mRNA, which results in an associated decrease in EHV-1 gB and EHV-1 Ori helicase protein synthesis.

The method comprises administering to a horse polynucleotides in the form of siRNAs or shRNAs that are designed to target EHV-1 gB and EHV-1 Ori helicase mRNA. Vectors encoding such shRNAs can also be used and are discussed in more detail below. It is expected that the method is applicable to any type of horse, including any type of horse that is at risk for or becomes infected with EHV-1. The method is also expected to be effective against any strain of EHV-1.

Specific examples of polynucleotide sequences suitable for use in the method are presented in Table 1. For Table 1, in the “Name” column, the “si” indicates an siRNA targeting either gB or Ori, as indicated. Numerals designate each particular siRNA tested. ORF33 encodes gB and ORF53 encodes the Ori helicase. Each of these ORFs can be discerned from the sequences presented in GenBank number AY665713 (Sep. 8, 2005 entry). For each siRNA shown, both the sense strand and the antisense strand are shown in the 5′ to 3′ direction. However, in a conventional double-stranded siRNA configuration, the sense strand and antisense strand will be anti-parallel and fully complementary to each other, but for a two nucleotide overhang at the end of each strand, as is typical for siRNAs. Bases that pair in each of the siRNAs in Table 1 are shown in capitals; the overhanging nucleotides are shown in lowercase, and may be deoxyribonuecleotides or ribonucleotides, or modified nucleotides. The sequence of each strand of an siRNA is at times referred to herein as an “siRNA polynucleotide” for convenience of reference. The siRNAs termed “sigB3” and “SiOri2” are preferred.

TABLE 1

| | Gene target | siRNA sequence |
|---|---|---|
| Name | (protein) | (5′ → 3′) |
| sigB1 | ORF33 (gB) | GGAUGGAGACUUUUACACCtt |
| | | (SEQ ID NO: 1) |
| | | GGUGUAAAAGUCUCCAUCCtc |
| | | (SEQ ID NO: 2) |
| sigB2 | ORF33 (gB) | GGAGAACGAGAUUUUCACGtt |
| | | (SEQ ID NO: 3) |
| | | CGUGAAAAUCUCGUUCUCCtc |
| | | (SEQ ID NO: 4) |
| sigB3 | ORF33 (gB) | CGGAAAUCGAGGUUAUCAGtt |
| | | (SEQ ID NO: 5) |
| | | CUGAUAACCUCGAUUUCCGtg |
| | | (SEQ ID NO: 6) |
| siOri1 | ORF53 | CGAUAACCUCCUCAACAAUtt |
| | (helicase) | (SEQ ID NO: 7) |
| | | AUUGUUGAGGAGGUUAUCGtc |
| | | (SEQ ID NO: 8) |
| siOri2 | ORF53 | CGAUGGUUCACCUCAACAAtt |
| | (helicase) | (SEQ ID NO: 9) |
| | | UUGUUGAGGUGAACCAUCGta |
| | | (SEQ ID NO: 10) |
| siOri3 | ORF53 | CGGAGGUUUUUGAAAACGAtt |
| | (helicase) | (SEQ ID NO: 11) |
| | | UCGUUUUCAAAAACCUCCGtc |
| | | (SEQ ID NO: 12) |
| siLuc | Firefly | Accession No: U47296 |
| | Luciferase | |
| siGFP | eGFP | Accession No: U55761 |

Each siRNA polynucleotide used in the invention can consist of between 21-29 nucleotides, inclusive, and including all integers between 21 and 29. It will be recognized by the skilled artisan that siRNA polynucleotide sequences of the invention that extend beyond 21 nucleotides and up to 29 nucleotides can be comprised of sequences that are identical, complementary to, or different from the EHV-1 gB mRNA or Ori helicase mRNA sequence, depending upon which gene is being targeted and whether the particular siRNA polynucleotide sequence is identical or complementary to the mRNA sequence expressed by that gene, and whether any non-EHV-1 encoded sequences are used.

In one embodiment, the method comprises administering a composition comprising an siRNA polynucleotide that comprises the sequence of SEQ ID NO:5 annealed to an RNA polynucleotide that comprises the sequence of SEQ ID NO:6, and an siRNA polynucleotide that comprises the sequence of SEQ ID NO:9 annealed to an siRNA polynucleotide comprising the sequence of SEQ ID NO:10. Each of these siRNA polynucleotides may also consist of the recited sequences.

In another embodiment, the method comprises administering to the horse a first shRNA polynucleotide targeted to EHV-1 gB mRNA and a second shRNA polynucleotide targeted to EHV-1 Ori helicase mRNA. As is known in the art, shRNAs adopt a typical secondary structure that contains a paired sense and antisense portion, and a short loop sequence between the paired sense and antisense portions. shRNA is delivered to the cytoplasm where it is processed by dicer into siRNAs. The siRNAs are recognized by RNA-induced silencing complex (RISC), and once incorporated into RISC, siRNAs facilitate cleavage and degradation of targeted mRNA. Thus, in the method of the invention, a first shRNA polynucleotide can comprise the sequence of both siRNA polynucleotides present in a double stranded siRNA molecule, such as an siRNA that is targeted to EHV-1 gB mRNA. Likewise, a second shRNA polynucleotide can comprise the sequence of both polynucleotides present in a double stranded siRNA molecule targeted to EHV-1 Ori helicase mRNA. Representative siRNA polynucleotide sequences suitable for inclusion in the shRNAs are presented in Table 1. Each of the shRNAs can consist of between 45-100 nucleotides, inclusive, and including all integers between 45 and 100.

In one embodiment, the method comprises administering a first shRNA polynucleotide that comprises SEQ ID NO:5 and SEQ ID NO:6, and simultaneously administering a second shRNA polynucleotide comprising the sequence of SEQ ID NO:9 and SEQ ID NO:10.

The method also comprises administering to the horse one or more viral vectors encoding a first shRNA polynucleotide targeted to EHV-1 gB mRNA and a second shRNA polynucleotide targeted to EHV-1 Ori helicase mRNA. Any viral vector capable of expressing the coding sequences for the shRNAs can be used. Examples of suitable vectors include but are not limited to viral based vectors, such as adenovirus (AV) vectors, adeno-associated virus (AAV) vectors, retroviral vectors [e.g, lentiviruses (LV) or murine leukemia virus], rhabdoviruses [rabies virus or vesicular stomatitis virus (VSV)].

The method of the invention can be performed by administering the polynucleotides as naked polynucleotides, or in combination with any suitable pharmaceutically acceptable carriers, excipients and/or stabilizers. Some suitable examples of pharmaceutically acceptable carriers, excipients and stabilizer can be found in Remington: The Science and Practice of Pharmacy (2005) 21st Edition, Philadelphia, Pa. Lippincott Williams & Wilkins.

The method can also be performed by administering the polynucleotides with a delivery agent. Suitable delivery agents include but are not limited to the Minis Transit TKO lipophilic reagent; lipofectin; lipofectamine; cellfectin; or polycations (e.g., polylysine), or liposomes.

In one embodiment, the polynucleotides are administered via intranasal administration, but the administration may be performed by any other suitable route. The polynucleotides can be delivered as a single dose or as multiple doses. The administrations can occur prior to exposure to EHV-1, during EHV-1 exposure, and after exposure to EHV-1, or in any combination of such time points. For example, the administrations can be performed at any point up to three days before an anticipated exposure, and/or during an exposure, and/or at any point for three days after symptoms of infection are no longer apparent. The method can be performed on horses that are at risk of coming into contact with EHV-1 so as to provide a prophylactic effect. Alternatively, horses that have become infected with EHV-1 can be treated so as to provide a therapeutic effect. One or more horses can be treated at or near the time of infection to provide a metaphylactic effect. Each of these effects can be evidenced by reduced severity of neurological symptoms that are induced by EHV-1 infection in horses, and/or by a reduction in EHV-1 viral shedding or viral load in the circulation (viremia) in the horses.

The polynucleotides can be delivered in conjunction with any conventional anti-viral treatment regimen, including administration of anti-viral agents, passive immunotherapies, vaccines, adjuvants, and the like.

The invention also provides compositions comprising polynucleotides that can inhibit EHV-1 gB and EHV-1 Ori helicase expression via RNAi mediated mRNA degradation. The polynucleotides can be any of those discussed supra as suitable for being used in the method of the invention, and accordingly include siRNAs, shRNAs, and vectors that encode shRNAs. Representative polynucleotide sequences that the compositions can comprises are set forth in Table 1. The compositions can comprise, consist essentially of, or consist of siRNAs, shRNAs, or viral vectors encoding shRNAs.

Polynucleotides for the compositions and methods of the invention can be made using any acceptable technique, including conventional and commercially available chemical synthesis techniques. The polynucleotides can also be expressed from expression vectors, and isolated and purified as necessary. Further, various types of polynucleotide modifications are contemplated so as to improve the capability of the polynucleotides to resist endonuclease degradation and/or improve cleavage of their target EHV-1 gene products. For example, in addition to RNA, the polynucleotides can comprise RNA:DNA hybrids. Other modifications that can be comprised by the polynucleotides include but are not limited to modified ribonucleotides or modified deoxyribonucleotides. Such modifications can include without limitation substitutions of the T position of the ribose moiety with an —O— lower alkyl group containing 1-6 saturated or unsaturated carbon atoms, or with an —O-aryl group having 2-6 carbon atoms, wherein such alkyl or aryl group may be unsubstituted or may be substituted, e.g., with halo, hydroxy, trifluoromethyl, cyano, nitro, acyl, acyloxy, alkoxy, carboxyl, carbalkoxyl, or amino groups; or with a hydroxy, an amino or a halo group. In addition to phosphodiester linkages, the nucleotides can be connected by a synthetic linkage, i.e., inter-nucleoside linkages other than phosphodiester linkages. Examples of inter-nucleoside linkages that can be used in the invention include but are not limited to phosphodiester, alkylphosphonate, phosphorothioate, phosphorodithioate, phosphate ester, alkylphosphonothioate, phosphoramidate, carbamate, carbonate, morpholino, phosphate trister, acetamidate, carboxymethyl ester, or combinations thereof.

The compositions and methods of the invention are shown to be capable of inhibiting EHV-1 infection in vitro as well as in a murine model of EHV-1 infection. Combining polynucleotides that target EHV-1 gB and EHV-1 Ori helicase is shown to elicit a synergistic inhibition of EHV-1 replication. The invention provides a demonstration of a reduction of the severity of neurological symptoms that are induced by EHV-1 infection in horses by using compositions and methods described herein. The invention also provides a demonstration of a reduction in EHV-1 plaque-forming units isolated from nasal secretions of horses two days after treatment, and therefore can be used to reduce EHV-1 viral shedding in infected horses. Further, performance of the method of the invention resulted in a reduction in severity of body temperature increases induced by EHV-1 infection. In particular, we observed in treated horses during days 6 through 7 after infection a marked, albeit non significant, reduction in severity of body temperature increases as compared to untreated control horses, wherein the latter experienced a second fever spike. It is also noteworthy that the method does not appear to adversely affect the humoral immune system of horses, as determined by a rise in titer appropriate for an acute infectious disease process in both the treated and untreated groups, and the lack of a significant difference in mean titers between the two groups after administration of the polynucleotides.

The following Examples are intended to illustrate but not limit the invention.

### Example 1

This Example provides a description of the materials and methods used to obtain the results presented in Examples 2 through 5.

Cells, Viruses and siRNAs. Rabbit kidney (RK13) cells were maintained in minimum essential medium (MEM, Mediatech Inc.) supplemented with 10% fetal bovine serum (FBS), 100 U/mL penicillin and 0.1 mg/mL streptomycin (Mediatech Inc.), at 37° C. under 5% CO₂ atmosphere. Wild type EHV-1 strain Ab4 and the eGFP-expressing rAb4Δgp2 were propagated in RK13 cells. The small interfering RNAs (siRNA's) against ORF33 (encoding gB) and ORF53 (encoding the helicase, Ori) were chemically synthesized (Ambion) based on the sequence of EHV-1 strain Ab4 (Genbank Sequence #AY665713) (Table 1).

siRNA Treatment and Virus Infection. Six-well plates of RK13 cells were treated with different concentrations of siRNA, ranging from 0 up to 75 pmol. The siRNA's were complexed with lipofectamine as per the manufacturers instructions (Invitrogen) and added to the cells in a total volume of 500 μL/well. After 2 hours of incubation at 37° C., 1.5 mL/well of growth media were added and incubated for 14 h. After washing, cells were inoculated with 100 plaque forming units (PFU)/well of eGFP-expressing rAb4Δgp2. One hour post infection (p.i.), medium was removed and infected cells were overlaid with fresh medium. Supernatants were collected at 24 h p.i. and cells were fixed with 10% formalin in phosphate buffered saline (PBS). To determine plaque sizes, at least 50 plaques per well were photographed and the average plaque areas were determined using the Image J software (rsb.info.nih.gov/ij). To determine extracellular viral titers, a standard plaque assay was used. Briefly, 10-fold dilutions of supernatants were plated on RK13 cells and 3 days p.i. cells were fixed with 10% formalin in PBS, stained with 0.3% crystal violet and plaques were counted. To evaluate the effectiveness of siRNA administration in relation to time of infection, siRNA's were added 12, 6 and 2 h prior to EHV-1 infection; simultaneously with EHV-1 infection; and 1 h after EHV-1 infection.

Western Blotting. Western blot analyses were performed conventional techniques. To detect gB, mAb 3F6 [Allen G P, Yeargan M R. J Virol 1987 August; 61(8):2454-61] was used at a 1:500 dilution. Anti β-actin (Sigma), at a 1:5000 dilution, was used as a control antibody. Anti-mouse IgG peroxidase conjugate was obtained from ImmunoResearch Laboratories and used at a dilution of 1:5000.

RNA extraction and real-time quantitative RT-PCR (qRT-PCR). Six-well plates of RK13 cells were transfected with siRNA and infected 14 h later with wild-type Ab4 as described above. At 12 and 24 h p.i. cells were collected and following a freeze-thaw cycle, RNA was extracted using RNA STAT-60 (Tel-Test Inc. Friendswood, Tex.) using standard methods. Briefly, dried RNA pellets were dissolved in 100 μl RNase-free water and all RNA samples were DNase-treated with the Turbo DNA-free kit, according to the manufacturer's instructions (Ambion). cDNA was synthesized using the Thermoscript RT-PCR system (Invitrogen) according to the manufacturer's instructions using random hexamers with Thermoscript reverse transcriptase. qPCR was performed using the 7500-FAST real-time PCR system (Applied Biosystems) with reaction mixtures containing TaqMan Fast Universal PCR Master Mix, 900 nM primers, 250 nM probe and 5 μL cDNA, in a 20 μl total reaction volume. Parameters included 95 C for 20 sec to activate Taq polymerase, followed by 40 cycles of 95 C×3 sec and 60 C×30 sec. The primers and probes used are listed in Table 2 and the comparative C_{T} method for relative quantitation (2^{-ΔΔCT}) was used with rabbit β-actin as the endogenous housekeeping gene.

Animal Experiments. All animal experiments in mice were performed in accordance with the U.S. Animal Welfare Act, under the supervision of Cornell University's Animal Care and Use Committee, and were conducted using established protocols. Three-to-four week-old female BALB/c mice (12 mice per group) were inoculated with varying concentrations and combinations of siRNA's and infected with 1×10⁵ PFU of the EHV-1 strain Ab4, as described in Table 3.

Suspensions of siRNA's, with or without the transfection reagent lipofectamine in 20 μL OptiMEM or PBS respectively, and Ab4 in 20 μL MEM were administered intranasally (Table 3). Individual weights of mice were determined daily on the day of infection (day 0) up to day 14 p.i. Three mice from each group were euthanized to collect lungs on days 2 and 4 p.i. The left lobes were homogenized to determine viral titers on RK13 cells by standard plaque assays. The right lobes were fixed in 10% formaldehyde solution and processed for histopathological analysis. H&E-stained lung sections of three mice per group were scored in a double-blinded manner under the light microscope to determine the degree of inflammation, bronchiolar changes (hyperplasia and/or necrosis) and the presence of inclusion bodies. A value of 0 (normal), 1 (minimal), 2 (mild), 3 (moderate), 4 (marked) or 5 (severe) was assigned for each histological parameter and the scores per group were computed as the total lung score per group. In another set of experiments, five mice per group were euthanized on day 3 p.i. to collect lungs for viral titrations and additional determination of the viral genome load in these tissues by qPCR, which was performed according to known methods [Allen G P, Yeargan M R. J Virol 1987 August; 61(8):2454-61].

Statistical Analysis. Student's t-test for paired data was used to test for differences. Data given are the means and bars show standard deviations. Body weights were compared using non-parametric Wilcoxon-Whitney and Kruskal Wallis tests. All statistical calculations were performed with SAS vs. 9.1. (SAS Corporation, Cary, N.C.).

### Example 2

This Example demonstrates that treatment with siRNA targeting glycoprotein B or the origin-binding protein helicase is effective in reducing EHV-1 replication in vitro.

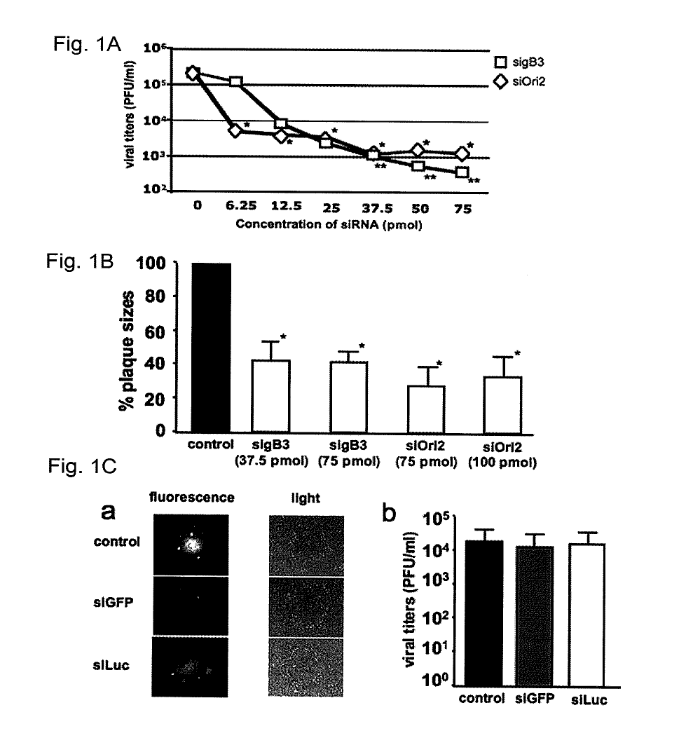

siRNAs directed against gB and On helicase were synthesized and evaluated for their effectiveness to reduce EHV-1 replication. sigB3 (see Table 1) directed against gB mRNA, efficiently reduced viral replication in a dose-dependent manner. A 10-fold reduction of viral titers was observed with as low as 6.25 pmol sigB3 (p<0.05) and reached a steady-state level starting at 37.5 pmol with up to a 100-fold reduction in viral titers (p<0.01) (FIG. 1A). Treating cells with SiOri2 (see Table 1), directed against the Ori helicase mRNA, resulted in a 50-fold reduction in viral titers, at a concentration ranging between 37.5 and 75 pmol (p<0.05). Furthermore, these siRNA's were also able to significantly reduce plaque sizes, with up to a 60% reduction in total plaque area when 37.5 pmol sigB3 or 75 pmol SiOri2 was used (p<0.05) (FIG. 1B).

To evaluate siRNA transfection efficiency, cells were transfected with commercially available siGFP, an siRNA targeting the egfp gene. eGFP-expressing EHV-1 strain rAb4Δgp2 was used for in vitro studies because eGFP expression allows for ready identification of infected cells and virus-induced plaques. Pre-treating cells with 75 pmol siGFP efficiently reduced eGFP expression in infected cells (FIG. 1C), without having any effect on EHV-1 infectivity, i.e. plaque sizes and titers remained unaffected even though eGFP expression was no longer detectable (FIG. 1C). To ensure that siRNA transfection by itself did not have a negative effect on EHV-1 infectivity, cells were treated with 75 pmol of the negative control siRNA siLuc, an siRNA targeting the luciferase gene, before infection. As expected, because the rAb4Δgp2 does not express luciferase, we did not observe any effect on eGFP expression (FIG. 1C) or on viral infectivity when compared to mock-treated cells (FIG. 1C).

To assess the effectiveness of sigB3 and SiOri2 at the mRNA level, relative quantitation of the two mRNA's was performed using quantitative reverse transcriptase PCR (qRT-PCR). Primers used for PCR amplifications are shown in Table 2.

TABLE 2
Real time PCR

| Gene | Primers (5′ → 3′)^{a} | MGB probe |
|---|---|---|
| EHV-1 IR6 | F: GCGAAGTACCCCTCGTTCATCT | TCGCGACACCGCCT |
| | (SEQ ID NO: 13) | (SEQ ID NO: 21) |
| | R: ATGCTCGGGCGCTCCTACT | |
| | (SEQ ID NO: 14) | |
| EHV-1 gB | F: CGCTGAGGATGGAGACTTTTACA | CCACCGCCTACCGGATCCACC |
| | (SEQ ID NO: 15) | (SEQ ID NO: 22) |
| | R: GGTGGTTCGATGCGTACG | |
| | (SEQ ID NO: 16) | |
| EHV-1 Ori | F: TGGTAACGGTGGGCCTTAGT | TTGATACGGCTCATT |
| | (SEQ ID NO: 17) | TCCACAGC |
| | R: GGGCTTGACGTAGGCAAACA | (SEQ ID NO: 23) |
| | (SEQ ID NO: 18) | |
| rabbit β-actin | F: CGAGATCGTGCGGGACAT | AAGGAGAAGCTGTGCTACGTG |
| | (SEQ ID NO: 19) | GCGCT |
| | R: GCCATCTCCTGCTCGAAGTC | (SEQ ID NO: 24) |
| | (SEQ ID NO: 20) | |

Sequencing

| Gene | Primers (5′ → 3′)^{a} |
|---|---|
| EHV-1 gB | F: TTTGGAAGCTGTGTTGTTAGAG |
| | (SEQ ID NO: 25) |
| | R: TAATCACTGCGGTATTGTCCA |
| | (SEQ ID NO: 26) |
| EHV-1 Ori | F: CACTTGCACCAGCCACGTTC |
| | (SEQ ID NO: 27) |
| | R: CATGGGGGTAAAGATGGGCT |
| | (SEQ ID NO: 28) |

^{a}F: Forward primer, R: Reverse primer.

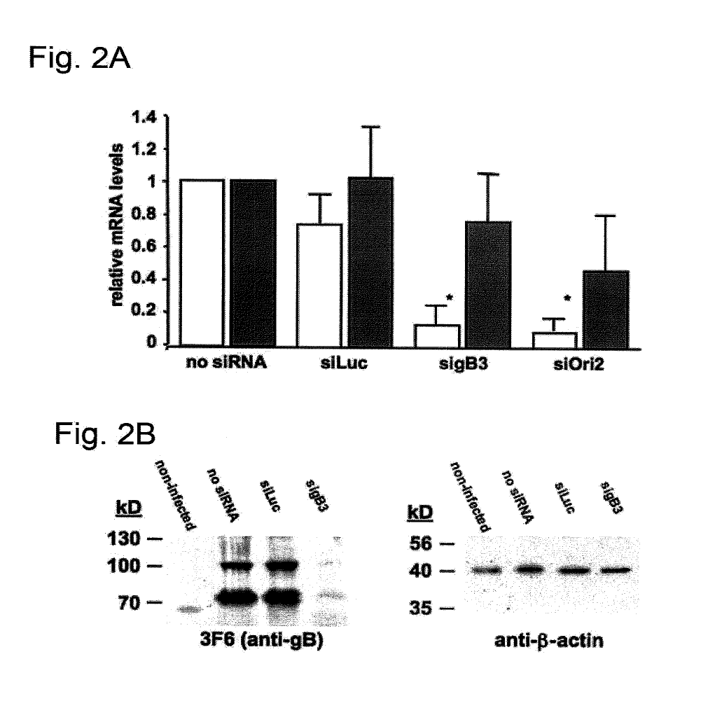

At 12 h p.i., the relative quantity of gB or Ori mRNA was reduced by more than 90% following sigB3 or Ori2 treatment, respectively, when compared to untreated cells (p<0.05) (FIG. 2A). No significant difference in gB or Ori mRNA levels in cells was observed between (i) untreated cells and cells treated with the control siRNA siLuc (FIG. 2A), and (ii) the control siRNA's siGFP and siLuc (data not shown). The effectiveness of silencing gB was also evaluated at the protein level by western blot analysis. Using the anti-gB antibody 3F6, a clear reduction in protein expression was observed in cells treated with sigB3 compared to non-treated cells or cells treated with control siLuc siRNA (FIG. 2B). Mock-infected cells did not express gB and the control antibody against β-actin showed that equal amounts of cell proteins were loaded in each lane (FIG. 2B).

We conclude from the data that siRNA treatment targeting EHV-1 gB or Ori can significantly reduce EHV-1 infection by effectively silencing gB or Ori expression at the mRNA and protein level. The silencing of these two essential EHV-1 proteins resulted in significantly reduced viral titers and plaque sizes.

### Example 3

This Example demonstrates that treatment with siRNA targeting gB3 and SiOri2 has a greater than additive effect on reduction of EHV-1 replication in vitro.

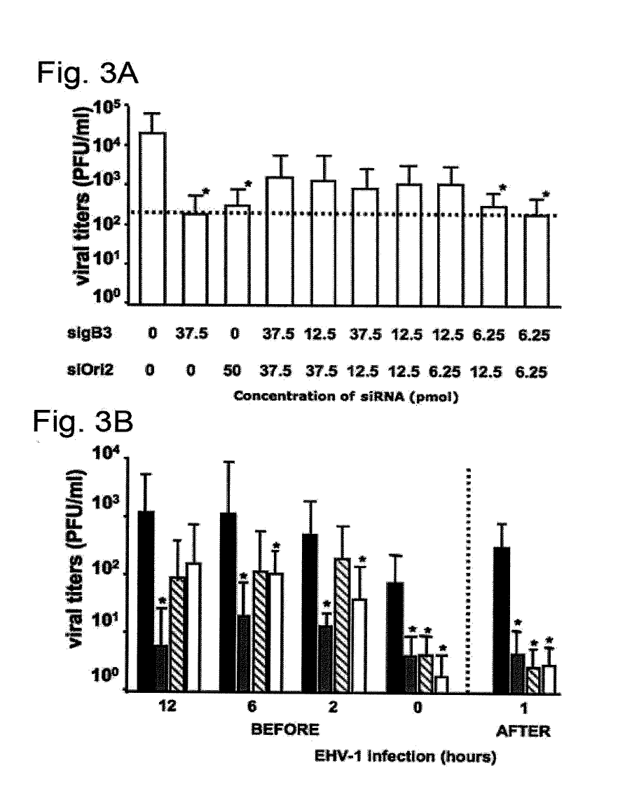

For this Example, we repeated the treatment described in Example 2, but used combinations of the two siRNA's. When applied together, gB3 and SiOri2 showed the same effectiveness in reducing EHV-1 infectivity, but at a much lower concentration than either siRNA by itself. As seen in FIG. 3A, the 80-fold reduction in viral titers observed after treatment with either 37.5 pmol sigB3 or 50 pmol SiOri2 was also obtained when using a combination of 6.25 pmol sigB3 and 6.25 pmol SiOri2 (p<0.05). Surprisingly, a combination of higher concentrations of sigB3 and SiOri2 (e.g. 37.5 pmol each or 12.5 pmol each) was less effective in reducing viral titers than the lower concentration of 6.25 pmol each (FIG. 3A). The effectiveness of this 6.25 pmol combination cocktail on gB silencing was also evaluated with both qRT-PCR and Western blot analysis and resulted in similar observations as described before with 37.5 pmol sigB3 (data not shown). These data indicate that targeting multiple genes simultaneously is as effective in reducing viral replication as when one gene is targeted, and does so with significantly lower concentrations of each of the individual siRNAs in a synergistic manner.

### Example 4

This Example demonstrates that siRNAs sigB3 and SiOri2, alone or in combination, also inhibit viral replication after infection. To obtain the data presented in this Example, cells were transfected with siRNA's at different time points before (12, 6 and 2 h), simultaneously, and 1 h after infection. It was observed that sigB3 (37.5 pmol) could significantly reduce viral replication when applied at any time between 12 h prior to and up to 1 h after infection (p<0.05) (FIG. 3B). SiOri2 at a concentration of 75 pmol reduced viral titers significantly when transfection and infection occurred simultaneously or when SiOri2 was transfected 1 h after infection (p<0.05) (FIG. 3B). The combination of sigB3 and SiOri2 at 6.25 pmol each, significantly decreased viral titers at all time points tested (p<0.05), with the exception of 12 h before infection (FIG. 3B). There was no significant difference at all time points tested between the control treatments that included cells treated with siGFP, siLuc or no siRNA (data not shown). Taken together, the in vitro experiments showed that siRNA treatment is not only effective in reducing EHV-1 replication when used before infection, but is as efficient, if not even more, when applied during or after the onset of EHV-1 replication.

### Example 5

This Example demonstrates that intranasal administration of siRNA reduces clinical sings and viral replication in a murine model of EHV-1 infection. The efficiency of siRNA therapy in vitro demonstrated in the preceding Examples clearly showed that cellular replication and spread of EHV-1 can be prevented in cell culture. A murine model was used to assess whether siRNA treatment is also effective in vivo. This mouse model of EHV-1 infection (Awan A R, et al. J Gen Virol 1990 May; 71 (Pt 5):1131-40), has been intensively used to predict the efficacy of putative EHV-1 vaccines in horses [Van Woensel P A, et al. J Virol Methods 1995 July; 54(1):39-49; Colle C F, III, T et al. Virus Res 1996 August; 43(2):111-24; Ruitenberg K M, et al. Vet Microbiol 1999 Aug. 16; 68(1-2):35-48; Ruitenberg K M, W et al Vaccine 1999 Jan. 21; 17(3):237-44], as well as the virulence potential of several EHV-1 strains. In the present case, mice were anaesthetized and inoculated intranasally with siRNA's, followed by infection with 1×10⁵ PFU EHV-1 Ab4 by the same route. The amounts and combinations of siRNA's used, as well as the time points of siRNA treatment and EHV-1 infection are summarized in Table 3.

TABLE 3

| | Concentration/combination of | Ttime of siRNA | Transfection | |
|---|---|---|---|---|
| Group | siRNA's (pmol) | application | reagent | Infection |
| A | sigB3 (187.5) | 24 h before I^{a} | lipofectamine | Ab4 |
| B | sigB3 (187.5) | 0.5 h before I | lipofectamine | Ab4 |
| C | siOri2 (187.5) | 24 h before I | lipofectamine | Ab4 |
| D | siOri2 (187.5) | 0.5 h before I | lipofectamine | Ab4 |
| E | sigB3/siOri2 (31.25/31.25) | 24 h before I | lipofectamine | Ab4 |
| F | sigB3/siOri2 (31.25/31.25) | 0.5 h before I | lipofectamine | Ab4 |
| G | sigB3/siOri2 (62.5/62.5) | 24 h before I | lipofectamine | Ab4 |
| H | sigB3/siOri2 (62.5/62.5) | 0.5 h before I | lipofectamine | Ab4 |
| I | siLuc (250) | 24 h before I | lipofectamine | Ab4 |
| J | no siRNA | 24 h before I | lipofectamine | Ab4 |
| K | siLuc (250) | 12 h before I | lipofectamine | Ab4 |
| L | siLuc (250) | 12 h before I | PBS | Ab4 |
| M | sigB3/siOri2 (62.5/62.5) | 12 h before I | lipofectamine | Ab4 |
| N | sigB3/siOri2 (62.5/62.5) | 6 h before I | lipofectamine | Ab4 |
| O | sigB3/siOri2 (62.5/62.5) | 12 h before I | PBS | Ab4 |
| P | sigB3/siOri2 (62.5/62.5) | 6 h before I | PBS | Ab4 |
| Q | sigB3/siOri2 (62.5/62.5) | 1 h after I | PBS | Ab4 |
| R | sigB3/siOri2 (62.5/62.5) | 6 h after I | PBS | Ab4 |
| S | sigB3/siOri2 (62.5/62.5) | 12 h after I | PBS | Ab4 |
| T | sigB3/siOri2 (62.5/62.5) | 24 h after I | PBS | Ab4 |
| control^{b} | no siRNA (PBS) | 24 h before | medium | medium |
| control | no siRNA (PBS) | 12 h before | medium | medium |

^{a}I: infection
^{b}control: to evaluate weight loss due to anesthesia.

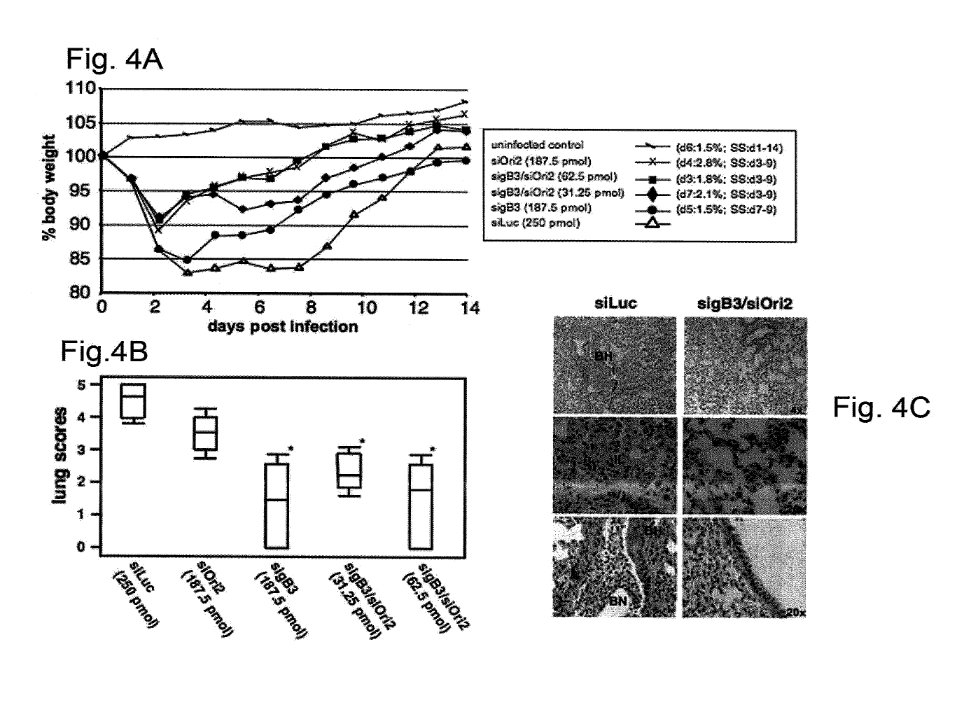

siRNA treatment with sigB3 and SiOri2, given alone or in combination at 24 h and 0.5 h before infection with EHV-1, was evaluated. Mice (12 per group) treated with the control siRNA siLuc began losing weight as early as 1 day p.i. and continued to lose weight until day 3 p.i., when a maximum weight loss of up to 17% of their original body weights was observed. Pre-infection weights were regained only at day 12 p.i. (FIG. 4A). These results were comparable to those obtained from mice that only received the siRNA transfection reagent lipofectamine before infection (data not shown). Mice treated with siRNA's targeting EHV-1 genes 0.5 h before infection also lost weight during the first 2 days p.i., but this weight loss only reached a maximum of 10% for the mice treated with 187.5 pmol SiOri2 and the combination sigB3/SiOri2 (31.25 pmol and 62.5 pmol, respectively). Mice treated with 187.5 pmol SiOri2 and 62.5 pmol sigB3/SiOri2 regained their original body weight by day 8 p.i., and mice treated with 31.25 pmol sigB3/SiOri2 by day 10 p.i. (FIG. 4A). The group treated with 187.5 pmol sigB3 lost up to 15% of their body weight and took until day 14 p.i. to regain their pre-infection weights. However, mice started regaining weight already by day 3 p.i., which is in contrast to the control siLuc treated group where the mice didn't start regaining weight until day 8 p.i. The differences between sigB3/SiOri2-treated mice and those treated with control siRNA's or transfection reagents alone were statistically significant on days 3-9 p.i. (non-parametric statistical testing, p<0.05) (FIG. 4A). The uninfected control group did not show any weight loss, indicating that the repeated anesthesia protocols used for the intranasal application of siRNA's and virus did not have any effect on weight loss (FIG. 4A). Since body weight loss caused by EHV-1 infection is strongly associated with an inflammatory response in lungs of infected mice, we validated the data on weight loss by histopathological analyses of lung tissues obtained on days 2 and 4 p.i. The lungs of mice treated with control siLuc had the most pronounced hyperplasia of bronchiolar epithelium at day 2 p.i., the most necrotic cells in the airway lumina, the most inclusion bodies as well as the most significant interstitial inflammation (FIGS. 4B and C). Lungs of mice treated with sigB3 were slightly less affected and lungs of mice treated with SiOri2 or the sigB3/Ori2 combinations were clearly less affected compared to the siLuc control group (FIGS. 4B and C).

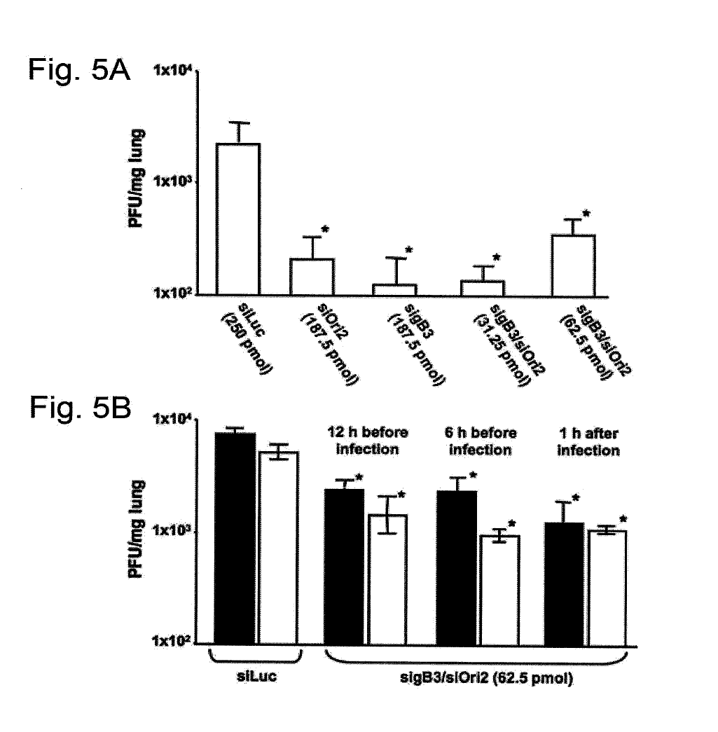

With regard to virus titers, mice inoculated with control siLuc had titers around 3×10³ PFU/mg lung tissue on day 2 p.i. (FIG. 5A). In contrast, virus titers in lungs of mice treated 0.5 h before infection with sigB3 and SiOri2, either alone or in various combinations, showed a significant reduction in viral titers with an average of 3×10² PFU/mg lung tissue (Student's t-test, p<0.05) (FIG. 5A). A similar reduction in virus titers was observed on day 4 p.i., although declining virus titers at that day generally indicates beginning clearance of the virus infection (data not shown). Treatment with siRNA's targeting EHV-1 genes at 24 h before infection also showed a reduction in body weight loss and virus titers to some extent when compared to the control siRNA group, albeit without reaching statistical significance. All infected mice showed maximal weight loss at day 4 p.i., with around 20% in the group of mice that received the control siLuc and only 15-17% in mice treated with siRNA's against EHV-1 genes. In addition, original body weights were regained 1 or 2 days earlier in the treated groups, compared to mice treated with control siLuc, in which pre-infection weights were only reached as late as 13 days p.i. Titers of 1.7±7.7×10³ PFU/mg lung tissue were observed in the siLuc-treated group, which were slightly higher than in lungs of mice treated with sigB3 (4.4±4.2×10² PFU/mg), SiOri2 (1.4±6.5×10³ PFU/mg) or the sigB3/SiOri2 combinations (1.6±1.3×10² PFU/mg and 1.3±1.0×10³ PFU/mg for 31.25 pmol sigB3/SiOri2 and 62.5 pmol sigB3/SiOri2 respectively). Taken together, this in vivo experiment revealed that siRNA therapy is effective in vivo by reducing clinical symptoms in challenge-infected animals and was capable of significantly reducing inflammation and viral replication in the target organ, the lung, at least when applied 0.5 h before infection.

Another experiment in mice was performed without a transfection agent. In addition, to extrapolate our previous findings to other time points, we included siRNA treatments 12 h and 6 h before infection with EHV-1. The siRNA combination of 62.5 pmol sigB3/SiOri2 was evaluated, since this treatment group was shown to significantly reduce both weight loss and virus replication. First, it was observed that intranasal application of sigB3/SiOri2 did not require a transfection reagent because no significant differences in weight loss (data not shown) and viral titers (Student's t-test, p<0.05) (FIG. 5B) were observed when siRNA's were delivered with either lipofectamine or PBS. Secondly, it was observed that siRNA treatment 12 h before infection was effective in significantly reducing both weight loss (non-parametric statistical testing, p<0.05, data not shown) and virus replication in the lungs (Student's t-test, p<0.05) (FIG. 5B). Treatment with 62.5 pmol sigB3/SiOri2 6 h before EHV-1 infection did not significantly reduce weight loss (data not shown); however, a significant reduction of virus titers in the lungs 2 days p.i. was observed in these mice (Student's t-test, p<0.05) (FIG. 5B).

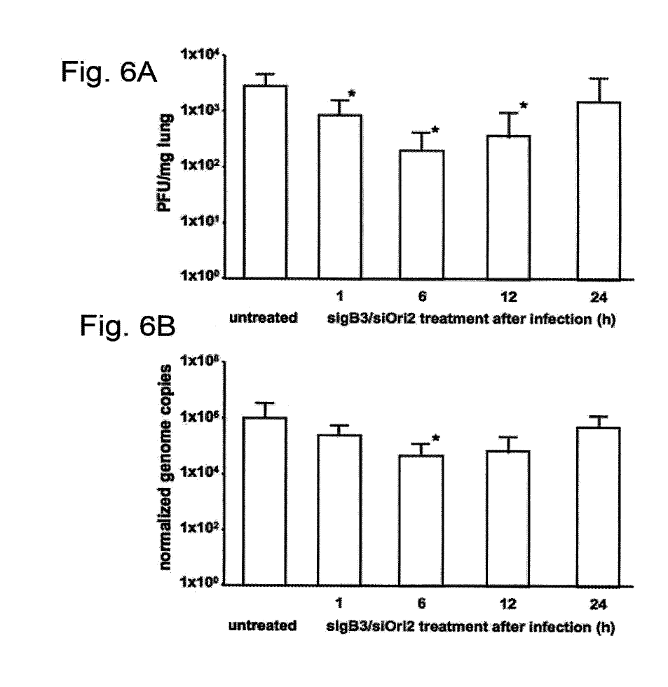

We also evaluated the effect of siRNA treatment after infection. Mice (15 per group) were infected with Ab4, followed by siRNA inoculation at 1, 6, 12 and 24 h after infection. The control group consisted of 15 untreated infected mice as we could never observe any significant differences between untreated and siLuc-treated mice in our previous experiments. Viral loads in lungs were determined by viral titrations and qPCR in five mice per group on day 3 p.i. The amount of infectious virus, as determined by virus isolation, in lungs of mice treated with siRNA's at 1, 6 and 12 h infection was significantly reduced (Student's t-test, p<0.05) compared to untreated infected animals (FIG. 6A). No significant reduction in virus titers was observed in animals treated with siRNA's 24 h post infection (p=0.9). A reduction in viral genome copies in the lungs of infected mice was observed at 1, 6 and 12 h p.i., but this reduction only reached statistical significance in the group of mice that were treated with siRNA's 6 h post infection (Student's t-test, p<0.05; FIG. 6B). These data show that treatment with siRNA's after infection is also efficient in reducing EHV-1 replication and therefore indicate the potential of siRNA treatment during an EHV-1 outbreak where several individuals might already have been exposed to the virus.

The foregoing Examples demonstrate that siRNAs directed against EHV-1 genes in vitro significantly reduce viral replication as measured by a significant reduction in both number and size of plaques. The results indicated that the siRNAs interfered with EHV-1 infectivity and were capable of decreasing virus spread to neighboring uninfected cells. Further, using the well-established in vivo murine model of EHV-1 infection, we were also able to demonstrate that mice treated intranasally with EHV-1-specific siRNAs were protected against clinical signs of infection, like weight loss. Importantly, the viral loads in the lungs of treated mice were significantly lower as assessed by (i) viral titration of lung tissues, (ii) quantitative real-time PCR and (iii) histological evaluation of inclusion bodies. In addition, histological evaluation of infected lung tissues of mice treated with control siRNAs directed against luciferase showed extensive perivascular cuffing as well as interstitial inflammatory inflammation. In contrast, significantly less inflammatory infiltrations and vascular changes were observed in mice treated with siRNA's that specifically target EHV-1 genes. We were also able to demonstrate the potential use of siRNA as a therapeutic alternative for the treatment of the animal respiratory viral disease caused by EHV-1 in a murine model. Besides showing the effectiveness of siRNA against EHV-1 when applied before infection, we demonstrated that siRNA administration to mice after challenge infection was effective in reducing clinical symptoms and virus replication in the lungs of treated animals. This might be of particular importance in the case of EHV-1 outbreak situations where in contact or previously exposed horses are treated. Based on these results, it is conceivable that if siRNAs are applied during an outbreak, not only the severity of clinical signs of affected horses but also the number of affected horses and the magnitude of nasal shedding could be reduced, resulting in an overall reduction of the viral load in the population and improved control of the outbreak.

### Example 6

This Example illustrates one embodiment of the invention and demonstrates reducing severity of neurological symptoms induced by EHV-1 infection in treated horses relative to neurological symptoms in untreated EHV-1 infected horses.

To obtain the data presented in this Example, horses were randomly assigned to treatment (10 horses) and control (4 horses) groups by an individual not involved in the clinical examinations or sample preparation and analysis. All other individuals involved in testing remained blinded until samples were fully processed. The protocol was approved by the Institutional Animal Care and Use Committee at Cornell University. Of the fourteen horses used in this experiment, 8 were geldings and 6 were mares, ranging in age from two to approximately 20 years of age. One mare of approximately 12 years old was determined to be pregnant on post-mortem examination. Breeds included Quarter Horse type, Standardbred, Arabian and Haflinger. The horses were kept in two different locations with access to pasture and free choice grass hay for a minimum of one month prior to the study. Previous vaccination history on the horses was unknown. All horses had negative Coggins tests (negative for equine infectious anemia virus). Serum neutralization titers were monitored on each horse at approximately monthly intervals.

Four days prior to initiating the experiment all horses were moved into a biosecurity level 2 isolation facility and allowed to acclimate during this time. Horses were maintained on free choice grass hay for the duration of the study. Blood samples and nasal swabs were obtained on days-1 (before infection), 1 through 10, 12, 14 and 21. Physical examinations including neurologic scoring were recorded twice per day for the first 48 hours post-infection, and on days 6 and 7, and daily on all other days. Neurologic scoring was performed by equine clinicians according to a commonly used clinical neurologic scoring system that primarily measures degree of ataxia (unsteadiness). The scoring system ranges from Grade 0 (normal horse) to Grade 5 (recumbent/unable to stand). Grades 1 through 4 are assigned based upon increasing severity and consistency of neurological deficits. On days when data was recorded once, horses were still observed by a veterinarian an additional time. Criteria for euthanasia prior to the end of the study were recumbence (horse was unable to stand on its own) or inability to perform basic bodily functions (eating, drinking, urination, defecation). The remaining horses were euthanized after day 21 so as to avoid introducing neurovirulent EHV-1 into the horse population at large.

The siRNAs administered were sigB3 and SiOri2 (see Table 1). Control horses received siRNA targeting firefly luciferase (siLuc).

On the morning of Day 0, treated horses received (750 pmols of each sigB3 and SiOri2) intranasally using an aerosol applicator. Control horses received 1,500 pmols of siLuc. Twelve hours later, all horses were infected with 1×10⁷ PFU EHV-1 strain Ab4 intranasally using the same technique of aerosol application. At twelve hours post-infection, the siRNA treatment was repeated exactly as described for the pre-infection application. All intranasal administrations were performed by the same two investigators for consistency of technique.

Samples obtained from each horse on days-1, 1 to 10, 12, 14 and 21 were placed on ice for transport between facilities and processed immediately as follows. Blood was drawn into two 10 ml sodium heparin tubes, one 10 ml serum tube, one 7 ml K3 EDTA tube and one 2 ml K3 EDTA tube. Aliquots of whole blood from one 10 ml heparin and one 7 ml EDTA tubes were refrigerated for later use. The remaining tubes were allowed to settle and the plasma layer containing peripheral blood mononuclear cells (PBMCs) was removed and added to tubes prepared with Ficoll solution. Tubes were centrifuged and the layer containing PBMC was removed, washed with PBS and resuspended in the buffer. The resuspended cells were applied immediately to wells containing RK13 cells maintained with minimum essential medium supplemented with fetal bovine serum and a penicillin, streptomycin, amphotericin B solution for virus isolation, and the remainder was frozen at −80° F. for subsequent qPCR processing.

Nasal swabs were obtained using two sterile, polyester tipped swabs and placed directly in 2 ml of viral transport medium. Samples were centrifuged (500×g, 10 min, 4 C) to remove debris, and applied immediately to RK13 cells maintained with minimum essential medium supplemented with fetal bovine serum and a penicillin, streptomycin, amphotericin B solution for virus isolation. The remainder was frozen at −80° F. for subsequent qPCR processing.

Cerebrospinal fluid samples were collected immediately following euthanasia. Aliquots were submitted for cytology, and the remainder was frozen at −80° F. for subsequent qPCR processing.

Post-mortem examinations were performed on all horses that developed neurological signs. Tissue samples were collected from the central nervous system, submandibular lymph nodes and the trigeminal ganglion, and frozen at −80° F. for subsequent qPCR processing.

DNA extraction of PBMCs, nasal swabs, CSF and tissues was performed using commercially available kits (E-Z 96 Blood DNA Kit, Omega Bio-tek; DNeasy 96 Blood & Tissue Kit Qiagen). qPCR was performed on frozen samples at the conclusion of the study using the 7500 FAST real time PCR system.

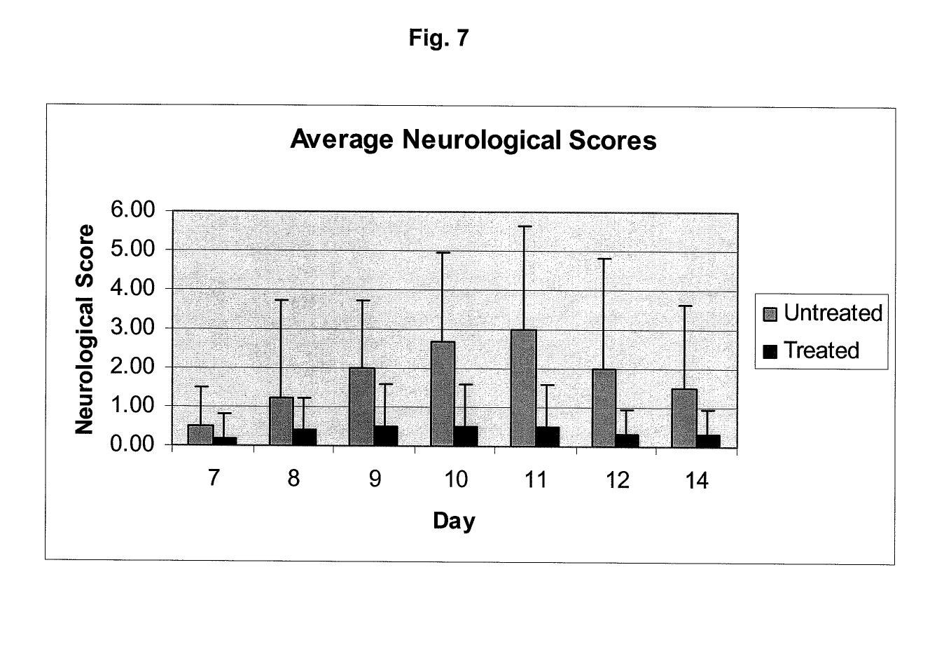

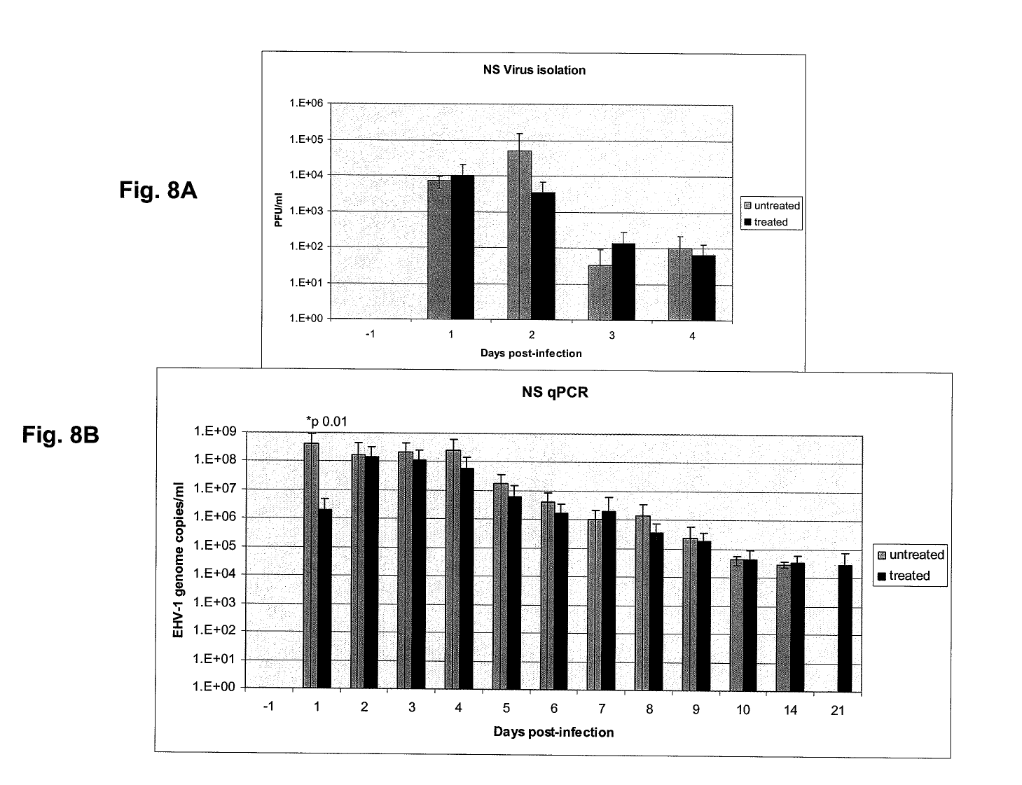

Data obtained from performance of the foregoing procedures are summarized in FIGS. 7 and 8. As can be seen from FIG. 7, a statistically significant reduction in the severity of neurological symptoms induced by EHV-1 infection in treated horses relative to neurological symptoms in untreated EHV-1 infected horses was observed at several time points during a 14 day period. Thus, these data demonstrate that the invention provides a method suitable for reducing the severity of neurological symptoms induced by EHV-1 infection in horses.

FIG. 8 provides a summary of data showing a decrease in EHV-1 PFU/ml from nasal swabs obtained from treated EHV-1 infected horses relative to untreated horses at the second day after infection and a significant decrease of virus genome copy numbers on day one after infection. Therefore, the invention can be used in methods for reducing EHV-1 viral shedding in EHV-1 infected horses.

While the invention has been described through illustrative examples, routine modifications will be apparent to those skilled in the art, which modifications are intended to be within the scope of the invention.

We claim:

1. A method for prophylaxis or treatment of EHV-1 infection in a horse comprising:
a) administering to the horse a composition comprising a first siRNA containing a polynucleotide comprising the sequence of SEQ ID NO:5 annealed to a polynucleotide comprising the sequence of SEQ ID NO:6, and a second siRNA containing a polynucleotide comprising the sequence of SEQ ID NO:9 annealed to a polynucleotide comprising the sequence of SEQ ID NO:10, wherein each of the polynucleotides in the first and second siRNA consists of between 21-29 nucleotides;
b) administering to the horse a composition comprising a first shRNA polynucleotide comprising the sequence of SEQ ID NO:5 and SEQ ID NO:6, and a second shRNA polynucleotide comprising the sequence of SEQ ID NO:9 and SEQ ID NO:10, wherein the first and second shRNA polynucleotides each consist of between 42-100 nucleotides; or
c) administering to the horse a composition comprising a vector encoding the first shRNA polynucleotide and the second shRNA polynucleotide, or a vector encoding the first shRNA polynucleotide and a distinct vector encoding the second shRNA polynucleotide.

2. The method of claim 1, wherein the first siRNA comprises a polynucleotide consisting of the sequence of SEQ ID NO:5 annealed to a polynucleotide consisting of the sequence of SEQ ID NO:6, and the second siRNA comprises a polynucleotide consisting of the sequence of SEQ ID NO:9 annealed to a polynucleotide consisting of the sequence of SEQ ID NO:10.

3. The method of claim 1, wherein the vectors are independently selected from the group of vectors consisting of adenovirus (AV) vectors, adeno-associated virus (AAV) vectors, retroviral vectors, and rhabdovirus vectors.

4. The method of claim 1, wherein the neurological symptoms induced by EHV-1 infection are selected from ataxia, recumbence, mild, moderate or complete paralysis, an inability to pass urine, and combinations thereof.

5. The method of claim 1, wherein performing a), b) or c) results in a reduction in severity of neurological symptoms induced by EHV-1 in the horse, relative to neurological symptoms in an EHV-1 infected horse for which none of a) b) and c) has been performed.

6. The method of claim 1, wherein performing a), b) or c) results in a reduction in severity of an increase in body temperature of the horse, relative to an increase in body temperature in an EHV-1 infected horse to which none of a) b) and c) have been performed.

7. The method of claim 1, wherein a) b) or c) is performed prior to the horse being exposed to EHV-1, during exposure to EHV-1, after the horse is exposed to EHV-1, or in combinations thereof.

8. The method of claim 7, wherein a) b) or c) is performed at least 24 hours prior to exposure to EHV-1.

9. The method of claim 1, wherein the composition comprising the first and second siRNA polynucleotide, or the first and second shRNA polynucleotide, or the vector encoding the first shRNA polynucleotide and the distinct vector encoding the second shRNA polynucleotide, further comprises a pharmaceutically acceptable carrier.

10. The method of claim 1, wherein the first and second siRNA polynucleotide, or the first and second shRNA polynucleotide, or the vector encoding the first shRNA polynucleotide and the distinct vector encoding the second shRNA polynucleotide, comprises a modified nucleotide and/or a modified inter-nucleoside linkage.

11. A composition comprising:
a) a first siRNA containing a polynucleotide comprising the sequence of SEQ ID NO:5 annealed to a polynucleotide comprising the sequence of SEQ ID NO:6; and/or a second siRNA containing a polynucleotide comprising the sequence of SEQ ID NO:9 annealed to a polynucleotide comprising the sequence of SEQ ID NO:10; wherein each of the polynucleotides in the first and second siRNA consists of between 21-29 nucleotides;
b) a first shRNA polynucleotide comprising the sequence of SEQ ID NO:5 and SEQ ID NO:6 and a second shRNA polynucleotide comprising the sequence of SEQ ID NO:9 and SEQ ID NO:10, wherein the first and second shRNA polynucleotides each consist of between 42-100 nucleotides; or
c) a vector encoding the first shRNA or the second shRNA from b).

12. The composition of claim 11, wherein the first siRNA polynucleotide consists of the sequence of SEQ ID NO:5 and is annealed to a polynucleotide that consists of the sequence of SEQ ID NO:6, and wherein the second siRNA polynucleotide consists of the sequence of SEQ ID NO:9 and is annealed to a polynucleotide that consists of the sequence of SEQ ID NO:10.

13. The composition of claim 11, further comprising a pharmaceutically acceptable carrier.
